(19) United States
(12) Patent Application Publication
(10) Pub. No.: US 2006/0258895 A1
(43) Pub. Date: Nov. 16, 2006

(54) Catheter, catheter device and imaging diagnostic apparatus

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/432,740

(22) Filed: May 11, 2006

(30) Foreign Application Priority Data
May 12, 2005 (DE) 10 2005 022 120.3

(51) Int. Cl. A61N 5/00

(52) U.S. Cl. 600/1

(57) ABSTRACT

The invention relates to a catheter for brachytherapy having a radiation source for generating β or γ rays. In order to be able to position the catheter as accurately as possible it is proposed according to the invention that a position indicating means is provided in the area of a free end of the catheter, by means of which position indicating means a position in a three-dimensional coordinate system can be determined on the basis of interactions produced when an external magnetic field is applied.

### CROSS REFERENCE TO RELATED APPLICATIONS

This application claims priority of German application No. 102005022120.3 filed May 12, 2005, which is incorporated by reference herein in its entirety.

### FIELD OF THE INVENTION

The invention relates to a catheter. It further relates to a catheter device as well as to an imaging diagnostic apparatus comprising the catheter or the catheter device.

### BACKGROUND OF THE INVENTION

A catheter of this type is known from WO 97/25102 A1. In practice the problem when using the known catheter is that the radiation source often cannot be positioned with the necessary precision therewith in the vessel that is to be treated.

In order to counteract this disadvantage, the known catheter has been combined with imaging apparatuses. These can be, for example, apparatuses in which the image is generated on the basis of ultrasound signals. An apparatus of this type is known for example from DE 198 27 460 A1.

WO 01/11409 A2 describes a catheter for optical coherence tomography (OCT). With the said apparatus, sectional images of the vessel surrounding the catheter are generated by means of infrared light. With this it is also possible in particular, using suitable computational methods, to produce a three-dimensional representation of a vessel being examined. Nonetheless, it has once again proven difficult in practice, after producing such a three-dimensional representation of the vessel, to arrange a radiation source precisely at a therapy position that is recognizable on the three-dimensional representation.

Furthermore, according to the prior art apparatuses are known for example from EP 0 776 176 B1, EP 1 034 738 B1 or EP 0 993 804 A1 by means of which the position of a catheter in the body can be determined. In such prior art solutions there are provided on a catheter a plurality of position indicating means, e.g. magnetic or electromagnetic transmitters or receivers, which interact with an external magnetic field. On the basis of the interactions it is possible to deduce a position of the position indicating means provided on the catheter in a three-dimensional coordinate system. Furthermore it is known from the above cited documents that the position data captured with the apparatus can be overlaid with image data captured by a further apparatus. The position of the catheter in an image generated on the basis of the image data can therefore be accurately represented. That said, however, the known apparatuses permit no treatment, more particularly of restenoses.

### SUMMARY OF THE INVENTION

The object of the invention is to eliminate the disadvantages according to the prior art. In particular a catheter, a catheter device and an imaging diagnostic system are to be specified by means of which restenoses in particular can be effectively and reliably treated.

This object is achieved by the features of the claims. Beneficial embodiments of the invention will emerge from the features of the claims.

According to the invention it is provided that in the area of the free end a position indicating means is provided by means of which a position in a three-dimensional coordinate system can be determined on the basis of interactions produced when an external field is applied. With the proposed catheter it is possible to arrange a radiation source for therapy purposes, in particular for treatment of restenoses, precisely at a predetermined position in the vascular system. This permits an improved therapy. The time required for treating a restenosis can be reduced.

The external field can be in particular a magnetic field. The magnetic field can be a magnetic or electrical alternating field. It is, however, also conceivable to detect the position indicating means using an ultrasound field. The position indicating means can comprise at least one, preferably three, coils. The coils can be provided with ferrite cores. In this way it is possible to generate and/or receive electromagnetic signals. The position indicating means can therefore be transmitters and/or receivers of electromagnetic signals.

According to a further embodiment it is provided that a magnetic field generated by at least two permanent magnets or coils has a different direction. The magnetic fields generated by the permanent magnets or coils are beneficially different from one another by at least 30°, and preferably 60° to 90°. In particular an arrangement of the permanent magnets or coils offset by 60° with regard to the direction of the magnetic fields allows on the one hand a miniaturization of the apparatus and on the other hand—using suitable computing algorithms—an accurate determination of the position of the position indicating means. For completeness, reference is made to the disclosure of the above cited EP 0 776 176 B1, EP 1 034 738 B1 or EP 0 993 804 A1, the contents of which are included herewith.

According to a further particularly advantageous embodiment there is provided in the area of a free end an OCT device for generating and capturing optical signals for producing optical coherence tomography first image data. An OCT device of this kind is generally known according to the prior art. In this regard reference is made to the above cited WO 01/11409 A2, the contents of which are included herewith.

The proposed combination of a catheter provided with a radiation source with a position indicating means and an OCT device enables an accurate visual display of the position of the catheter, for example in a three-dimensional image.

According to a further advantageous embodiment it is provided that in the area of the free end there is provided an ultrasound device for generating and capturing ultrasound signals for producing second image data. An ultrasound device of this kind is generally known according to the prior art. In this regard reference is made, for example, to the above cited DE 198 27 460 A1 and to R. J. Dickinson, “Miniature ultrasonic probe construction for minimal access surgery”, Phys. Med. Biol. 49 (2004), pages 3527 to 3538. The disclosure of these documents is included herewith by reference.

Precisely the combination of the catheter according to the invention with the ultrasound device is particularly advantageous, since ultrasound signals have a greater penetration depth into the tissue than optical signals and furthermore an examination is also possible when an x-ray contrast agent is made to flow through the vascular system.

According to a further advantageous embodiment it is provided that in the area of the free end there is provided an inflatable balloon. The balloon enables a vessel surrounding the catheter to be closed. It also enables the catheter to be held at a particular position in the vascular system. In this way it is possible to inject a rinsing fluid into the vessel through the catheter in order to be able subsequently to record first image data by means of the OCT device and to reconstruct first images therefrom.

It has also revealed itself as advantageous to attach deflection means in the area of the free end. The deflection means can comprise at least one, preferably more than one, further permanent magnets and/or electromagnets. In this case a magnetic field generated by at least two further permanent magnets and/or electromagnets can have a different direction. With the proposed deflection means it is possible to deflect the free end of the catheter in a desired direction upon application of suitable external magnetic fields. This facilitates the guiding of the free end of the catheter on a predetermined path into the vessel that is to be treated.

The position indicating means as well as, where applicable, the radiation source, the OCT device and/or the ultrasound device and/or the deflection means can be attached to an elongated carrier structure forming the area of the free end, which structure is connected to a flexible tube. The stiffness of the carrier structure is beneficially greater than the stiffness of the tube. The result of this is that the free end of the catheter touches a wall of a vessel surrounding the catheter as often as possible. As a consequence thereof a particularly accurate calculation of the position and/or a path of the position indicating means can be performed from the data obtained with the position indicating means.

In the context of the present invention a “free end of the catheter” is understood to mean an end which is advanced into the vascular system as far as the vessel that is to be treated. The free end beneficially has a rounded-off shape in order to avoid an injury to the vascular system. An “area of the free end” describes a section which comprises the free end of the catheter and which is necessary for accommodating in particular the position indicating means, where applicable the OCT device, the ultrasound device, the deflection device and suchlike. The area of the free end comprises in particular the carrier structure. It is typically 1 cm to 5 cm long.

According to a further embodiment it is provided that a layer surrounding the carrier structure and/or the tube is provided for shielding magnetic fields. The layer can contain hollow fibers produced from an electrically conductive material or nanomagnetic particles. In this way in particular signal lines can be shielded against magnetic fields which are generated in particular externally or by the coils.

According to a further embodiment it is provided that the carrier structure and/or the tube are/is provided with a marker which can be recognized during the production of the x-ray image. This enables the position determined with the position indicating means to be correlated with image data for an x-ray image assigned to a further coordinate system.

A transponder indicating the property of the catheter can also be provided. This enables the remote interrogation of specific properties of the catheter. In addition parameters relating to an appropriate control of the catheter can be transmitted wirelessly to an external system. Finally information can be stored in the transponder to enable the catheter to be tracked in a logistics chain of a clinic.

There is additionally provided in accordance with the invention a catheter device having an outer catheter movably guided on an inner catheter, at the end of which outer catheter a radiation source is attached, and wherein there is provided at the free end of the inner catheter and/or at the end of the outer catheter a position indicating means by means of which a position in a 3-dimensional coordinate system can be determined on the basis of interactions produced when an external field is applied. With the proposed alternative solution it may in particular be that no radiation source is provided on the inner catheter. The alternative solution initially enables the inner catheter to be introduced into the vessel that is to be treated. The inner catheter can then be used as a sort of guiding means and the outer catheter can be pushed as far as the vessel that is to be treated. The outer catheter has the radiation source at its end. A certain amount of time is required in order to reach the vessel to be treated with the inner catheter. A radiation exposure taking place during this time can be reduced through the provision of an outer catheter having a radiation source attached at its end.

According to an advantageous embodiment it is provided that the radiation source is embodied as an annular or hollow cylinder so that the inner catheter can be pushed back and forth through the radiation source. This enables the outer catheter to be introduced subsequently into the vessel. This enables the advantages already mentioned above to be achieved.

The outer catheter, like the inner catheter, can also be provided with a marker which can be recognized during the production of an x-ray image. In addition, a layer surrounding the outer catheter can be provided for shielding magnetic fields. The layer can in turn contain hollow fibers produced from an electrically conductive material and/or nanomagnetic particles. Reference is made to that extent to the above cited advantages.

The inner catheter can, incidentally, have the same embodiment features as the outer catheter. To that extent reference is made to the preceding statements.

Also provided according to the invention is an imaging diagnostic apparatus having an inventive catheter or an inventive catheter device, wherein a device is provided for determining a position indicated by the position indicating means in the three-dimensional coordinate system. Devices for determining the position of a position indicating means are known according to the prior art. These can be electromagnetic transmitters or alternatively electromagnetic receivers which interact with the position indicating means. Depending on the embodiment, the position indicating means can be either transmitters or receivers. In general, at least one transmitter is assigned to a receiver or vice versa. For the sake of completeness reference is made in this context to the disclosure of the following, above cited, documents included herewith: EP 0 776 176 B1, EP 1 034 738 B1 or EP 0 993 804 A1.

Advantageously the device for determining the position has at least two field generators for generating magnetic fields, in particular magnetic alternating fields of different frequency. This enables the location of the position indicating means to be established in the three-dimensional coordinate system.

According to a further embodiment of the invention, a device for calculating a vessel center line representing the path of the position indicating means is provided. Said line is a 1-dimensional line in the three-dimensional coordinate system. It can be described with reference to the coordinates which can be determined by the position of the position indicating means using a polynomial equation. For completeness reference is made in this context to the disclosure of U.S. Pat. No. 6,546,271, which is included herewith.

According to a further embodiment, a means is provided for calculating a vessel envelope describing a vessel wall. This enables for example a minimum and a maximum vessel diameter to be estimated and vessel constrictions to be detected.

According to a further embodiment, a device is provided for rotating the OCT device and/or the ultrasound device. This enables for example a glass fiber running in the catheter and/or an ultrasound sensor to be rotated at a constant speed about the catheter axis. As a result exposures of a vessel surrounding the catheter can be made circumferentially in the area of the free end of the catheter. The signals supplied by the OCT device and/or the ultrasound device can be evaluated in the device for rotating. Toward that end the device for rotating can include a device for evaluating the OCT signals and/or ultrasound signals. In this case the evaluation primarily comprises a digitization of the captured signals as well as their correlation with a specific angle of rotation.

According to a further embodiment, the imaging diagnostics apparatus can have a means for generating a two- or three-dimensional image using the positions determined with the position indicating means and using the first and/or second image data. The means for generating a two- or three-dimensional image is advantageously a computer by means of which the image and position data can be correlated using a suitable image reconstruction program and processed to produce an image representing the vessel. At the same time image artifacts can be corrected in particular using the vessel center line determined with the position indicating means.

According to a further embodiment of the invention, a means is provided for correlating the coordinates determined using the position indicating means with further coordinates. An x-ray apparatus having at least one semiconductor detector and a data processing device can be provided for determining the further coordinates. With the proposed embodiment it is possible, for example, to correlate images which were generated with further imaging diagnostic apparatuses with the images supplied using the inventive catheter or catheter device. Furthermore a device can be provided for optional overlaying of a first image/images determined on the basis of the first image data and/or a second image/images determined on the basis of the second image data and/or a third image generated using an imaging diagnostic device. Such an overlaying or fusion leads to images having a high informational value. They can be, for example, three-dimensional images in which the position of the catheter can be accurately represented.

The imaging diagnostic device can be selected from the following group: x-ray apparatus, preferably x-ray computer tomograph; magnetic resonance tomograph; positron emission tomograph; endoscopic image acquisition device.

According to a further embodiment, a device is provided for generating an external magnetic field of predetermined direction and strength for the purpose of deflecting the deflection means. With the proposed device, the imaging diagnostic apparatus is expanded by the further function of a precise guidance of the catheter in the vascular system. For this purpose the deflection means provided in the area of the free end of the catheter can be selectively deflected using directed external magnetic fields and in this way a change in direction of the free end of the catheter to a predetermined direction can be achieved.

In particular in the field of angiography it has proven advantageous to combine the proposed imaging diagnostic apparatus with what is referred to as a “bi-plane x-ray apparatus”, in which at least one x-ray source, a first semiconductor detector arranged in a first plane and a second semiconductor detector arranged in a second plane different from the first plane are provided. This enables large-area survey exposures to be produced on which the position of the catheter in the vascular system can be recognized, in particular on the basis of the x-ray markers provided thereon.

### BRIEF DESCRIPTION OF THE DRAWINGS

Exemplary embodiments of the invention are explained in more detail below with reference to the drawings, in which:

FIG. 1 shows a schematic cross-sectional view of a catheter,

FIG. 2 shows a schematic cross-sectional view of a first catheter device,

FIG. 3 shows a schematic cross-sectional view of a second catheter device,

FIG. 4 shows a schematic cross-sectional view of an inner catheter,

FIG. 5 shows a schematic cross-sectional view of a third catheter device,

FIG. 6 shows a schematic cross-sectional view of a fourth catheter device,

FIG. 7 shows a schematic overview of the essential components of an imaging diagnostics apparatus, and

FIG. 8 shows a schematic representation of a method for producing a three-dimensional image.

### DETAILED DESCRIPTION OF THE INVENTION

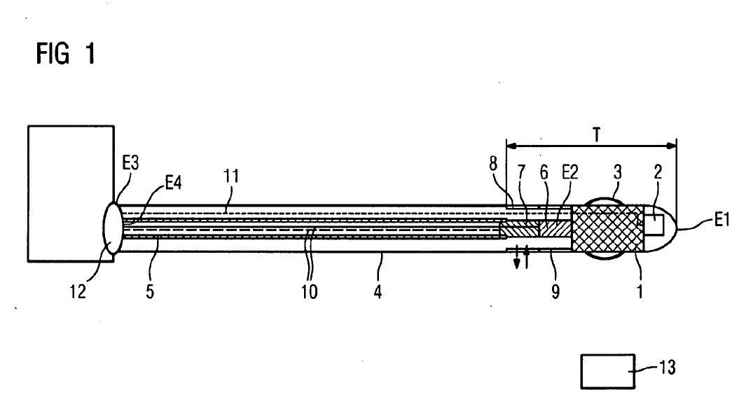

In the catheter shown in FIG. 1, a free end E1 is embodied as a rounded shape. In the area of the free end E1 the catheter is provided with a radiation source 1 by means of which β or γ radiation can be generated for therapeutic purposes.

Position indicating means are designated by the reference numeral 2. Said means can be, for example, three coils which are arranged offset by 90° to one another in the X, Y and Z direction. However, the coils can also be arranged relative to one another under a different offset angle, for example 60°. Instead of the coils, other suitable transmitting or receiving means, permanent magnets for example, can also be provided in an appropriate offset arrangement with regard to the direction of the magnetic flux. The reference numeral 3 denotes an inflatable balloon.

Accommodated in a tube 4 of the catheter is a core 5 which is rotatable about a catheter axis and at the first end E2 of which an ultrasound transducer 6 and a transmitting/receiving device for optical signals 7 (=OCT sensor) are attached. The OCT sensor 7 is provided opposite a window 8 that is transparent to optical signals, and the ultrasound transducer 6 is arranged opposite a further window 9 that is transparent to ultrasound signals. A carrier structure T accommodating in particular the radiation source 1, the position indicating means 2 and the window 8 as well as the further window 9 extends over an area which includes the free end E1. The carrier structure T can be produced for example from a plastic. It advantageously has a greater stiffness than the tube 4.

Supply and/or signal lines 10 connected to the ultrasound transducer 6 and the OCT sensor 7 are integrated in the core 5. Further supply and/or signal lines 11 connected to the position indicating means 2 are provided in the tube 4 or on an internal wall of the same.

A third end E3 of the tube 4 and a fourth end E4 of the core 5 are accommodated in a rotation device 12. The tube 4 is held secure in the rotation device 12, while the core 5 can be rotated with the rotation device 12. The connection can be made using a rotation coupling which enables a supply voltage and/or signals to be coupled in and/or out.

The reference numeral 13 identifies a transmitting/receiving device which is arranged outside of a body that is to be examined. This enables a position of the position indicating means 2 in a three-dimensional coordinate system to be determined mathematically, using a computer for example, and displayed, by means of a monitor for example.

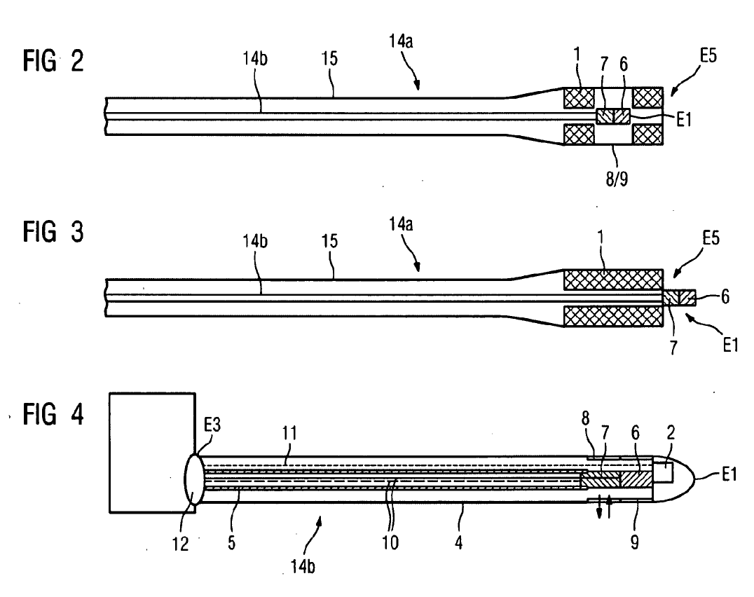

FIGS. 2 and 3 show catheter devices in which a radiation source 1 is attached at a further free end E5 of the outer catheter 14a. In this arrangement the radiation source 1 is implemented in the form of rings or a hollow cylinder. The outer catheter 14a has a further tube 15. An inner diameter of the further tube 15 and a diameter of the rings or the hollow cylinder are embodied such that an inner catheter 14b, as shown for example in FIG. 4, can be guided through them. The proposed catheter arrangement therefore consists of an inner catheter 14b which is movably guided in the outer catheter 14a.

The inner catheter 14b shown in FIG. 4 is similar in structure to the catheter shown in FIG. 1. In this case only the balloon 3 and the radiation source 1 are omitted.

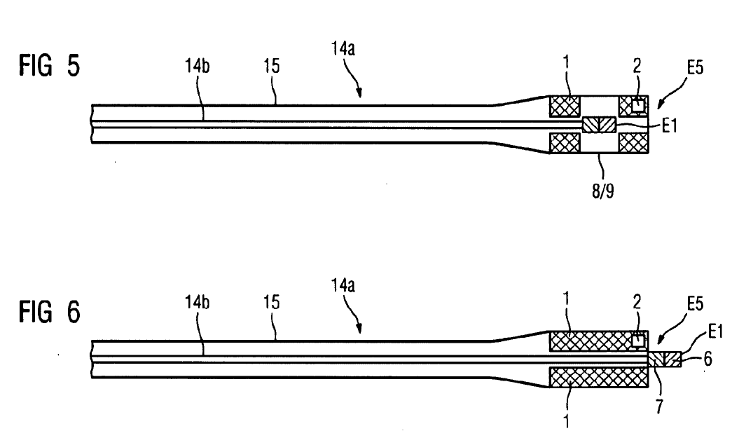

In the further catheter arrangement shown in FIGS. 5 and 6, the position indicating means 2 is attached in the area of a further free end E5 of the outer catheter 14a. In this case the position indicating means 2 can be omitted in the inner catheter 14b. It is, however, also possible that in the further catheter arrangement according to FIG. 5 or 6 an inner catheter 14b according to FIG. 4 is used. In this case the outer catheter 14a can also be provided with a further position indicating means which supplies distinguishable signals compared to the position indicating means 2. As a result of this, a position of the outer catheter 14a in the three-dimensional coordinate system can be determined separately by means of the further position indicating means.

The further catheter arrangement shown in FIGS. 5 and 6 is in turn characterized in that an inner catheter 14b, embodied for example according to FIG. 4, is guided movably therein, while a free end E1 having the OCT sensor 7 and the ultrasound transducer 6 can be guided through a passage provided at the further free end E5 of the outer catheter 14a.

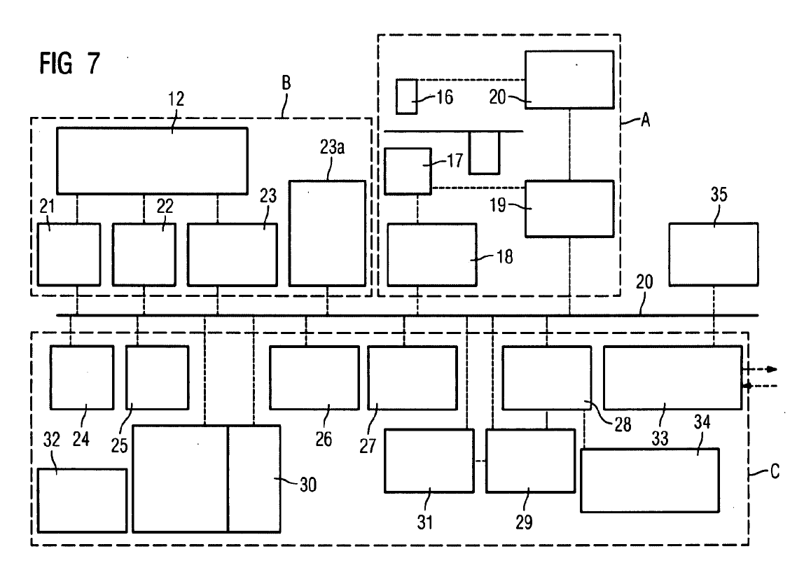

FIG. 7 shows the main components of an imaging diagnostic apparatus. The imaging diagnostic apparatus essentially consists here of an x-ray apparatus A, a catheter control and signal acquisition device B and a powerful data processing device C.

The x-ray apparatus A includes an x-ray emitter 16, one or more x-ray detectors 17, an x-ray image processing unit 18, an x-ray control apparatus 19 and a high-voltage generator 20. The x-ray image processing unit 18 and the x-ray control apparatus 19 are connected to a data bus 20.

The catheter control and signal acquisition device B has the rotation device 12 already described in FIG. 1 for the purpose of connecting a catheter (not shown here). The rotation device 12, in which a digitization of the supplied data can already take place, is coupled to an OCT image processing unit 21 and an ultrasound image processing unit 22 as well as a position signal processing unit 23. In order to reduce movement artifacts caused, for example, by a patient's breathing or by the movement of a patient's heart, sensors detecting such physiological functions can be provided. The reference numeral 23a designates a unit for recording and processing signals supplied by physiological sensors. The aforementioned units are in turn connected to the data bus 20.

A powerful data processing device C supports parallel processing, in particular image processing, of the data supplied via the data bus 20. Thus, for example, the data processing device C can comprise a first image processing device 24 for producing OCT images, a second image processing device 25 for producing images from ultrasound signals, a third image processing device 26 for producing images from position signals, a fourth image processing device 27 for producing x-ray images, an image fusion and image reconstruction unit 28, an image correction unit 29, as well as a display and operator control unit 30 for displaying the generated images. The image correction unit 29 can be connected to the data bus 20 via a calibration unit 31. The reference numeral 32 identifies a voltage supply and the reference numeral 33 designates an interface for importing and exporting patient data. The reference numeral 34 designates a database in which parameter data of the x-ray emitter or of a β, γ emitter is stored. Finally, the reference numeral 35 designates a data memory which is used in particular for storing image data.

The following typical method sequence can be implemented with the proposed imaging diagnostic apparatus in combination with the proposed catheter or the catheter device:

Introduction of the catheter or the inner catheter under x-ray control, with contrast agents optionally being used;

Generation of a survey radiographic, more particularly angiographic, exposure;

Generation of images using the position indicating means;

Generation of images using the ultrasound transducer and/or the OCT sensor;

Overlaying of the images generated using the position indicating means and the images generated by means of radiographic methods;

Overlaying of the images generated using the ultrasound transducer and/or the OCT sensor with images generated by means of radiographic methods;

Three-dimensional reconstruction of the images obtained by means of the ultrasound transducer and/or OCT sensor using the images obtained with the position indicating means;

Navigation of the catheter or the inner catheter on the basis of the generated images as far as the target position;

Inflation of the balloon at the target position and rinsing with rinsing fluid;

Generation of high-resolution images by means of the ultrasound transducer and/or the OCT sensor in the area of the target position;

Where applicable, introduction of an outer catheter as far as the target position, said outer catheter being pushed over the inner catheter;

Monitoring of the exact position of the outer catheter by means of the ultrasound transducer and/or OCT sensor and/or the position indicating means.

The provision of the position indicating means in particular enables three-dimensional images to be produced from the signals supplied by the OCT sensor and ultrasound transducer. It is, for example, possible, after a survey angiographic exposure has been produced, to represent the path of the catheter through suitable use of the signals supplied by the position indicating means exclusively by means of the signals supplied by the ultrasound transducer 6 and/or OCT sensor 7 as well as the signals supplied by the position indicating means 2 and thereby reduce the patient's exposure to x-rays. The proposed imaging diagnostic apparatus supplies important, more particularly accurate, medical information on, for example, the arteriosclerotic plaque and/or tumor tissue. Aside from this, the location of the free end of the catheter can be precisely determined.

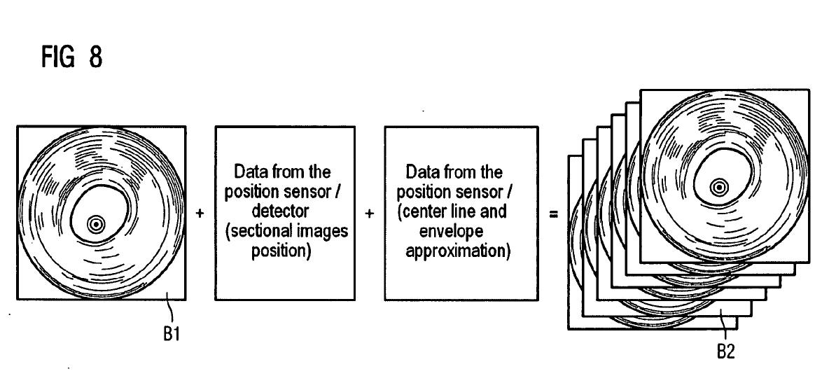

FIG. 8 shows in schematic form the production of a corrected volume data set using the position data obtained with the position indicating means 2. The signals obtained using the OCT sensor 7 and/or the ultrasound transducer 6 can be processed to provide two-dimensional first images B1. The first images B1 can also be produced by a fusion of images obtained with the OCT sensor 7 and the ultrasound sensor 6. The first images B1 thus generated can subsequently be corrected using the position data supplied by the position indicating means 2. Toward that end, the data obtained with the position indicating means 2 can be reconstructed mathematically, for example according to the discrete tomography method described, for example, in DE 102 24 011 A1, and a three-dimensional image can be computed therefrom.

Furthermore, a center line of the vessel and/or an envelope of the vessel can be computed from the data supplied by the position indicating means 2. Using these computational models the first images B1 can then be processed to produce a set of second images B2 which have reduced artifacts in relation to the first images B1.

In order to register or, as the case may be, overlay the patient's image data with the data obtained using the position indicating means 2, it is necessary to transfer the spatial coordinates of the image data and the position data into a common coordinate system. In the process movements of a patient during the examination can cause errors. A magnetic auxiliary sensor, as described, for example, in U.S. Pat. No. 6,233,476 B1, can be used for the purpose of error correction. An auxiliary sensor of this kind can also be provided cablelessly, e.g. with a Bluetooth transmitter unit. Alternatively, a movement of the patient can also be captured by means of an optical camera and determined using mathematical methods of pattern recognition and taken into account in the registration of the image data.

Separate, generally known functional units can be provided in order to reduce movement artifacts which are caused, for example, by a patient's breathing or by the movement of a patient's heart.

1-38. (canceled)

39. A catheter for brachytherapy, comprising:
a radiation source attached in an area of a free end of the catheter for generating β or γ rays; and
a position indicator attached in the area for indicating a position of the catheter in a three-dimensional coordinate system so that the position is determined based on an interaction with an external magnetic, electrical, or ultrasound field.

40. The catheter as claimed in claim 39,
wherein the external field is a magnetic field,
wherein the position indicator comprises a magnet or coil that generates a further magnetic field which interacts with the external magnetic field,
wherein if the position indicator comprises a plurality of magnets or coils that generate a plurality of magnetic fields, then the plurality of magnetic fields have different direction with respect to each other.

41. The catheter as claimed in claim 39,
wherein a first image data is produced by an optical coherence tomography device for generating and capturing optical signals,
wherein a second image data is produced by an ultrasound device for generating and capturing ultrasound signals.

42. The catheter as claimed in claim 39, wherein an inflatable balloon is provided in the area of the free end.

43. The catheter as claimed in claim 39,
wherein a deflection device is attached in the area of the free end,
wherein the deflection device comprises a further permanent magnet or electromagnet,
wherein a plurality of magnetic fields generated by a plurality of further permanent magnets or electromagnets have different direction with respect to each other.

44. The catheter as claimed in claim 39, wherein the position indicator, or the radiation source, or the optical coherence tomography device, or the ultrasound device, or the deflection device is attached to a carrier structure in the area of the free end connected to a flexible tube and the stiffness of the carrier structure is greater than the stiffness of the tube.

45. The catheter as claimed in claim 44,
wherein the carrier structure or the tube is provided with a marker which is recognized during a production of an x-ray image,
wherein a layer surrounding the carrier structure or the tube is provided for shielding magnetic fields,
wherein the layer contains hollow fibers formed from an electrically conductive material or nanomagnetic particles.

46. The catheter as claimed in claim 39, wherein a property of the catheter is indicated by a transponder.

47. A catheter device, comprising:
an inner catheter;
an outer catheter which is movably guided by an inner catheter;
a radiation source attached toward an end of the outer catheter; and
a position indicator attached toward a free end of the inner or outer catheter for indicating a position of the inner or outer catheter in a three-dimensional coordinate system based on an interaction with an external magnetic, electrical, or ultrasound field.

48. The catheter device as claimed in claim 47, wherein the radiation source is a ring or hollow cylinder, the ring or hollow cylinder adapted to push the inner catheter back and forth through the radiation source.

49. The catheter device as claimed in claim 47,
wherein the outer catheter is provided with a marker which is recognized during a production of an x-ray image,
wherein a layer surrounding the outer catheter is provided for shielding magnetic fields,
wherein the layer containing hollow fibers formed from an electrically conductive material or nanomagnetic particles.

50. An imaging diagnostic apparatus for brachytherapy, comprising:
a catheter; and
a position determining device operatively connected to the catheter for determining a position of the catheter indicated by a position indicator in a three-dimensional coordinate system.

51. The imaging diagnostic apparatus as claimed in claim 50, wherein the position determining device has a plurality of field generators for generating magnetic alternating fields of different frequency.

52. The imaging diagnostic apparatus as claimed in claim 50,
wherein a first calculator is provided for calculating a vessel center line representing a path of the position indicator,
wherein a second calculator is provided for calculating a vessel envelope describing a vessel wall.

53. The imaging diagnostic apparatus as claimed in claim 50,
wherein a rotation device is provided for rotating the catheter for generating and capturing an optical or ultrasound signal,
wherein the rotation device comprises an evaluator for evaluating the optical or ultrasound signal.

54. The imaging diagnostic apparatus as claimed in claim 50, wherein a two- or three-dimensional image is generated using a position indicated by the position indicator and using a first or second image data.

55. The imaging diagnostic apparatus as claimed in claim 50,
wherein a coordinate system determined with the position indicator is correlated to a further coordinate system,
wherein the further coordinate system is determined by an x-ray apparatus which has a semiconductor detector and a data processing device.

56. The imaging diagnostic apparatus as claimed in claim 50, wherein an image is generated by overlaying a first image generated based on the first image data or a second image generated based on the second image data or a third image generated by an imaging diagnostic device.

57. The imaging diagnostic apparatus as claimed in claim 50, wherein a device for generating an external magnetic field of a predetermined direction and strength is provided for deflecting a deflection device.

58. The imaging diagnostic apparatus as claimed in claim 50, wherein an x-ray source comprises a first semiconductor detector arranged in a first plane and a second semiconductor detector arranged in a second plane which is different from the first plane.
